(19) United States
(12) Patent Application Publication
(10) Pub. No.: US 2016/0206819 A1
(43) Pub. Date: Jul. 21, 2016

(54) Anesthetic Syringe

(71) Applicant: John Alan Garrett, Tulsa, OK (US)

(72) Inventor: John Alan Garrett, Tulsa, OK (US)

(21) Appl. No.: 14/730,053

(22) Filed: Jun. 3, 2015

Related U.S. Application Data
(60) Provisional application No. 62/105,084, filed on Jan. 19, 2015.

(51) Int. Cl. A61M 5/24; A61M 5/315; A61M 5/31

(52) U.S. Cl. CPC A61M 5/24; A61M 5/3129; A61M 5/3137; A61M 5/3135; A61M 5/31565; A61M 2205/19

(57) ABSTRACT

An improved syringe has an enclosure which holds a carpule, with the enclosure having a first opening and a second opening. A needle is attached at the first opening via luer lock, while a plunger is inserted into the second opening. Pushing the plunger into the enclosure actuates the carpule and results in contained medication being dispensed from the carpule through the needle. A window is provided for the enclosure, allowing a user to easily view the carpule and aspirate. For improved ergonomics, the plunger has a handle while the enclosure has a grip enhancement. The handle is a dome shaped piece that comfortably receives a user's palm or thumb. The grip enhancement is a pair of wings, with each wing positioned on either side and contoured to allow a user's fingers to grasp the wings.

The current application claims a priority to the U.S. Provisional Patent application Ser. No. 62/105,084 filed on Jan. 19, 2015.

### FIELD OF THE INVENTION

The present invention relates generally to a non-traditional disposable anesthetic syringe that is sterile, compatible with standard carpules, and housed in an opaque medial-grade plastic casing to minimize anxiety in patients while still providing a means to observe aspirate.

### BACKGROUND OF THE INVENTION

Syringes and needles are utilized across a variety of medical professions in order to administer medications and anesthetics, both general and local. Unfortunately, many people are uncomfortable in the presence of needles, with some experiencing a fear of needles. In order to reduce these fears and improve patient comfort it is desirable to conceal as much of a needle as possible. However, a person (e.g. dentist) using the needle to administer anesthetic still must be able to view a carpule portion of the present invention in order to watch for aspirate.

It is therefore an object of the present invention to conceal a needle within an opaque body while still allowing for a carpule to be viewed in order to detect aspirate. It is a further object of the present invention to provide a sterile, modular, and user-friendly apparatus which can easily be assembled, cleaned, and disposed.

The present invention has been provided in order to address the needs of dentists administering local anesthetics, but ultimately the present invention is applicable to any environment where a needle and pre-fabricated carpules are used to administer a fluid to a person.

### BRIEF DESCRIPTION OF THE DRAWINGS

FIG. 1 is a top plan view showing an enclosure of the present invention.

FIG. 2 is a front elevational view showing the enclosure of the present invention.

FIG. 3 is a perspective view showing the enclosure of the present invention.

FIG. 4 is a front elevational view showing a plunger of the present invention.

FIG. 5 is a perspective view showing the plunger of the present invention.

FIG. 6 is a front elevational view showing the enclosure, plunger, and a needle of the present invention.

FIG. 7 is an internal front elevational view showing the plunger engaged with a carpule stored in the enclosure of the present invention.

FIG. 8 is a perspective view showing the enclosure, plunger, and needle of the present invention.

### DETAIL DESCRIPTIONS OF THE INVENTION

All illustrations of the drawings are for the purpose of describing selected versions of the present invention and are not intended to limit the scope of the present invention.

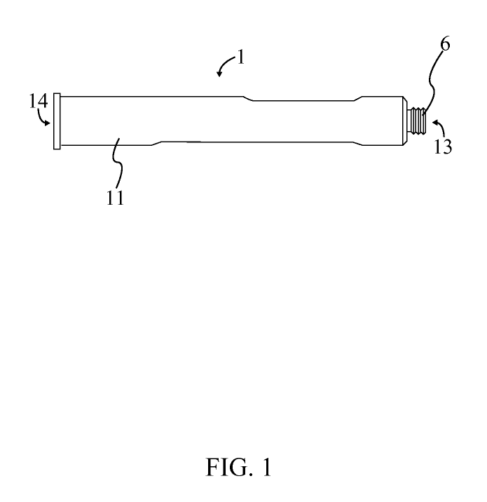

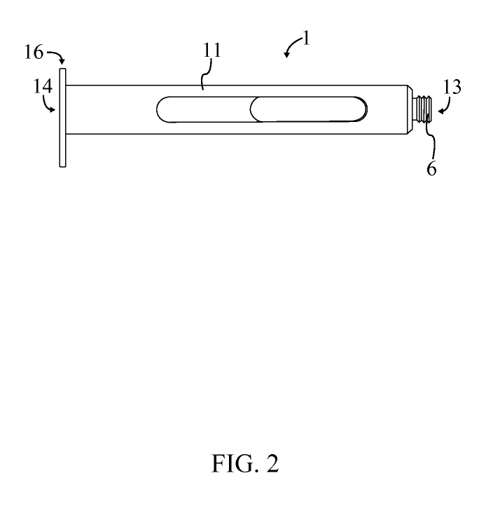

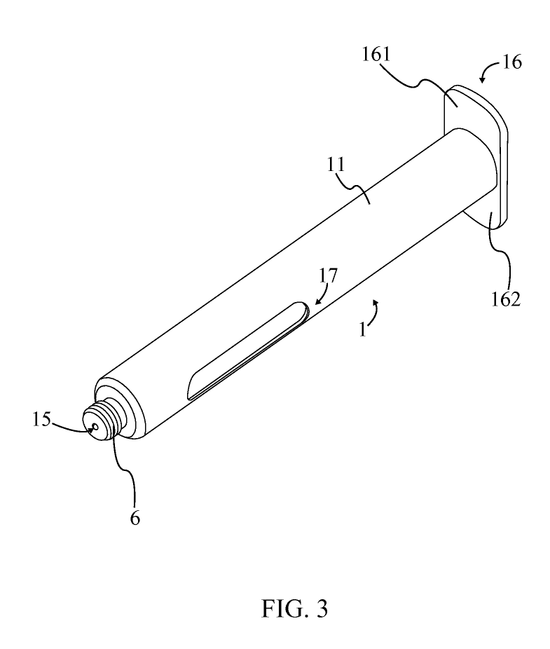

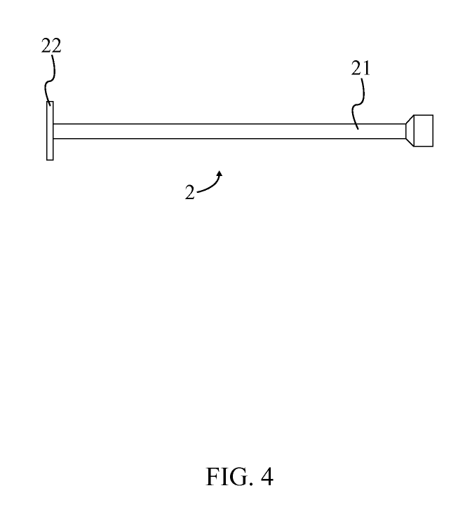

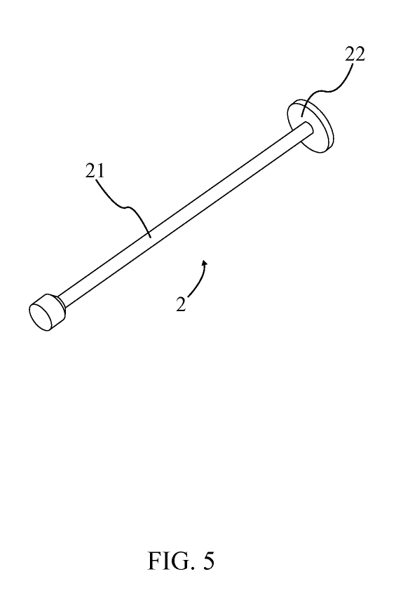

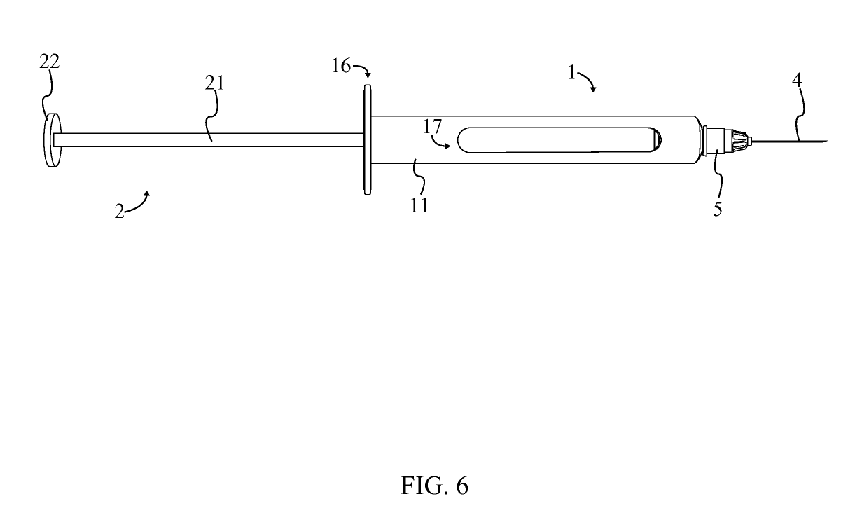

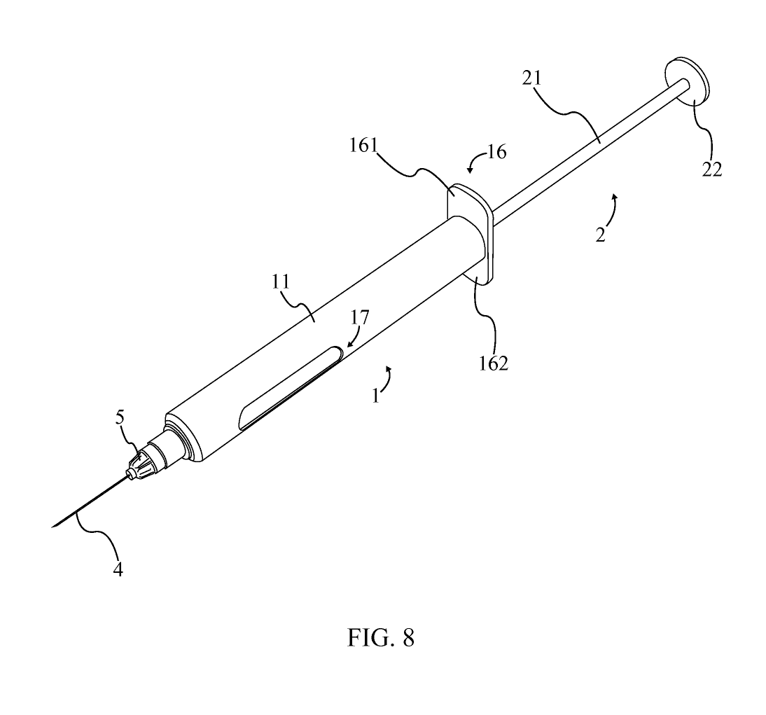

The present invention is an improved anesthetic syringe with carpule 3. The present invention comprises an enclosure 1, a plunger 2, and a carpule 3, with the carpule 3 being placed in the enclosure 1 and engaged by the plunger 2. More specifically, the enclosure 1 comprises an elongated body 11 within which a chamber 12 is formed. The chamber 12 is accessible at each end by a first opening 13 and a second opening 14, with the first opening 13 and second opening 14 being positioned opposite each other along the elongated body 11. The carpule 3 is placed in the chamber 12, such that the plunger 2 can be pressed against the carpule 3 in order to dispense medication via a connected needle 4. The needle 4, which pierces the carpule 3, is in fluid communication with the carpule 3 and thus allows a user to easily administer medication. The enclosure is individually illustrated in FIG. 1-FIG. 3, the plunger in FIG. 4 and FIG. 5, and the present invention as a whole in FIG. 6-FIG. 8. While a variety of materials are possible for the construction of the present invention, a plastic or otherwise disposable material is preferred due to the intended one-use nature of the present invention. This is in comparison to existing syringes which are not intended to be disposable.

The first opening 13 and the second opening 14 allow the needle 4 and the plunger 2, respectively, to be engaged with the enclosure 1. The chamber 12, which is positioned within the elongated body 11, is accessible at either end so that the needle 4 can enter the chamber 12 from the first opening 13 while the plunger 2 can enter the chamber 12 from the second opening 14. The aperture of the first opening 13 and the second opening 14 is variable, with each aperture preferably being sized to securely accommodate the corresponding needle 4 and plunger 2.

The profile of the chamber 12 is reduced at a neck portion of the enclosure 1 in order to limit motion of the carpule 3. The neck portion supports the carpule 3 such that the plunger 2 is capable of actuating the carpule 3. A conduit 15 is formed between the first opening 13 and the chamber 12, with the needle 4 traversing through the conduit 15 into the chamber 12. The conduit 15 is wide enough to allow passage of the needle 4 while narrow enough to prevent passage of the carpule 3. In this sense the combination of the conduit 15 and elongated body 11 creates a floor which supports the carpule 3 when the present invention is being used to dispense a contained medium through the needle 4.

Preferably, the enclosure 1 comprises a window 17 as shown in the illustrated embodiment. The window 17 is positioned on the elongated body 11, adjacent to the chamber 12. The window 17 allows a user to visually inspect the interior of the chamber 12 along with a loaded carpule 3; this is beneficial for viewing aspirate, as later described. Potentially, multiple windows 17 could be incorporated into the present invention (as with the illustrated embodiment), though only a single window 17 is necessary to allow viewing of the carpule 3 within the chamber 12.

In another potential embodiment, in place of a window 17, the enclosure 1 can be constructed from a transparent material, such that the elongated body 11 effectively acts as a window 17. The entire chamber 12 and carpule 3 is thus visible from all angles. This improves visibility compared to the illustrated embodiment in which a window 17 is provided. In another potential embodiment the window 17 is entirely omitted, though such an embodiment effectively negates the self-aspirating aspect of the present invention.

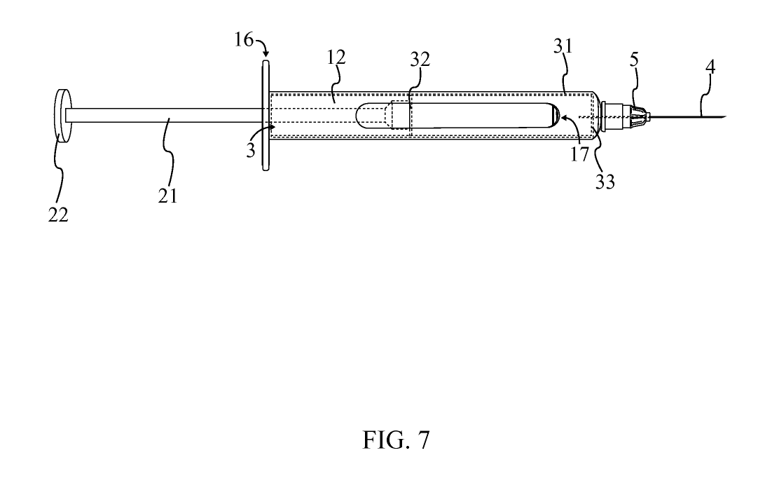

The carpule 3 comprises a hollow body, a plug 32, and a sealing membrane 33. The plug 32 and sealing membrane 33 are positioned at opposite ends of the hollow body, ensuring that a substance such as a medication is sealed within the hollow body. The plug 32 is a rigid component that is provided as an interactive surface for the plunger 2; thus the plunger 2 is positioned adjacent to the plug 32, allowing the plunger 2 to press upon the plug 32. The plug 32 is slidably engaged with the hollow body; as a result, pressure from the plunger 2 causes the plug 32 to move into the hollow body. This movement reduces the volume of the hollow body, creating an increase in pressure. The increased pressure, in turn, causes the medication to move through a needle 4 that is connected into the hollow body. The needle 4 itself is positioned at a dispensing end of the enclosure 1, with the needle 4 traversing into the chamber 12 through the first opening 13 of said enclosure 1. The components of the carpule 3 and positioning of the carpule 3 relative to the enclosure 1 and needle 4 are shown in FIG. 7.

The carpule 3 is loaded into the enclosure 1 prior to use, with the sealing membrane 33 being punctured by the aforementioned needle 4. The carpule 3 is easily loaded into the enclosure 1, allowing users to quickly load the desired carpule 3 immediately prior to use. The carpules 3 can thus be stored in a sterile environment until needed. This results in the present invention being preloaded, increasing convenience for end users.

In the preferred embodiment, the carpule 3 is a 1.8 cc dental anesthetic carpule 3 as known in the art; in other embodiments the carpule 3 can contain different amounts of medication, while the enclosure 1 is scaled up or down in size to fit the carpule 3 (if necessary).

Likewise, the needle 4 itself is provided with a coupling body 5, preferably of the luer-type, that allows the needle 4 to easily be attached to the enclosure 1 prior to use. The ease of attaching and detaching needles 4 means that users can quickly dispose of used needles 4 and replace said used needles 4 with sterilized needles 4. Effectively, the present invention can be stored in a disassembled configuration, with needle 4, enclosure 1, carpule 3, and plunger 2 being independent from one another. This modular assembly results in a user friendly apparatus that is kept sterile and is low cost to maintain. The low cost of maintenance results in part from the ability to reuse the enclosure 1 and plunger 2, only requiring a user to discard the needle 4 and carpule 3 after use, though the whole of the present invention can be considered disposable. Costs can further be reduced by later sterilizing the needle 4 such that it suitable for medical use again, as compared to outright disposal of the needle 4.

To ensure that the needle 4 remains sterile prior to use, it can be covered by a sterile paper during storage. This will prevent contamination until the needle 4 is ready for use, at which point the sterile paper is removed and the needle 4 is attached to the enclosure 1 via the coupling body 5.

The coupling body 5 encircles the needle 4, with a matching thread pattern 6 being positioned on the coupling body 5 and on the first opening 13 of the enclosure 1. The matching thread pattern 6 allows the coupling body 5 to be screwed into the first opening 13; i.e., the thread pattern 6 of the coupling body 5 becomes helically engaged with the thread pattern 6 of the first opening 13. This exemplifies a “luer lock” style connector. Alternatively, a “slip tip” style connector can be used; the slip tip style omits the thread pattern 6 from both the coupling body 5 and the first opening 13, instead attaching the coupling body 5 to the enclosure 1 by means of a press fit. In other words, the “slip tip” style uses friction to hold the coupling body 5 and enclosure 1 together. The above description serves to elaborate upon possible attachment means of the luer-type and does not preclude the use of other attachment means with the present invention. The coupling body 5 is illustrated in FIG. 6-FIG. 8.

In different embodiments of the present invention the needle 4 may be angled with respect to the enclosure 1, rather than being straight as shown in the illustrated embodiment. An angled needle 4 may be preferable to some users as it can make administering medication to oral areas easier; ultimately, the present invention functions the same independent of needle 4 shape and can thus be used with a variety of needle 4 designs.

The plunger 2 itself comprises an elongated portion 21 and a handle 22, which are adjacently connected to each other. The elongated portion 21 traverses into the chamber 12 and is positioned adjacent to the carpule 3. The handle 22 is positioned adjacent to the second opening 14, exterior to the enclosure 1. The handle 22 is preferably wider than the chamber 12 of the elongated body 11, such that the handle 22 is prevented from moving into the chamber 12. This helps to prevent the plunger 2 from completely traversing into the enclosure 1 and becoming stuck. The increased size of the handle 22 also makes the plunger 2 easier for a user to grasp thanks to increased surfaces area resulting from the higher width of the handle 22. A head is positioned opposite the handle 22 along the elongated portion 21, with the head being the portion of the plunger 2 that makes contact with and actuates the carpule 3, as shown in FIG. 7.

The handle 22 is provided as an ergonomic surface for grasping and interacting with the plunger 2. In the illustrated embodiment the handle 22 is implemented as a rounded dome shape structure that comfortably fits into a user's palm or easily pressed by a user's thumb. The shape of the handle 22 is not restricted to such, and in other embodiments different style handles 22 can be used in place of aforementioned rounded handle 22. Potentially, the handle 22 could even be omitted entirely, though such an omission would greatly reduce user friendliness of the present invention.

The enclosure 1 further comprises a grip enhancement 16, which allows a user to better grasp and operate the present invention in combination with the handle 22 of the plunger 2. In the illustrated embodiment, the grip enhancement 16 comprises a first brace 161 and a second brace 162 which are contoured to receive a user's fingers. A user can place their fingers around each brace, thus holding the enclosure 1 steady while their palm presses down on the handle 22 of the plunger 2. The combination of the grip enhancement 16 and the handle 22 allows a user to easily push the plunger 2 into the plug 32 of a carpule 3, resulting in the contents of the carpule 3 being dispenses via a connected needle 4. As earlier referenced, in other embodiments of the present invention the implementation of the handle 22 and grip enhancement 16 can be altered as compared to the illustrated embodiment while still allowing the plunger 2 to press the plug 32 of the carpule 3, as is necessary to dispense medication using the present invention. The contours of the grip enhancement 16 and the handle 22 allow for a user to hold the present invention with a variety of grips.

The present invention, as mentioned, can easily and quickly be assembled immediately prior to use. The assembly process it outlined below. First, the needle 4 is attached to the enclosure 1 by engaging the coupling body 5 of the needle 4 with a corresponding coupling body 5 of the enclosure 1, the latter coupling body 5 being positioned at the first opening 13 of the enclosure 1. In the preferred embodiment this is accomplished with the luer lock connection earlier referenced. Once the needle 4 is attached the carpule 3 is pierced as the needle 4 punctures the sealing membrane 33. The plunger 2 is used to press down upon the plug 32 of the carpule 3, causing the plug 32 to move into the carpule 3 body and resultantly forcing contained medication out of the carpule 3 via the needle 4.

Prior to dispensing the medication, a user is able to look through the window 17 to take advantage of the self-aspirating construction of the present invention. The window 17, which provides an unobstructed view of the carpule 3, enables a user to see if any blood is visible, the appearance of which indicates the needle 4 has been placed into a blood vessel. The user can then reposition the needle 4 so that the medication is not dispensed into the bloodstream. This ensures that the medication is locally administered as intended by the user.

Although the invention has been explained in relation to its preferred embodiment, it is to be understood that many other possible modifications and variations can be made without departing from the spirit and scope of the invention as hereinafter claimed.

1. An improved anesthetic syringe comprises:
an enclosure;
a plunger;
a carpule;
a needle;
the enclosure comprises an elongated body, a chamber, a first opening and a second opening;
the first opening and the second opening being positioned opposite each other along the elongated body;
the chamber being positioned within the elongated body between the first opening and second opening;
the carpule being housed within the chamber;
the plunger traversing into the chamber;
the plunger being positioned adjacent to the carpule;
the needle being in fluid communication with the carpule;
the needle being connected to a coupling body;
the needle being encircled by the coupling body;
the coupling body being attached to the elongated body;
the needle traversing through the first opening into the chamber;
the needle being positioned adjacent to the carpule;
the first opening and the coupling body each comprise a thread pattern;
the thread pattern of the first opening being helically engaged with the thread patter of the coupling body;
the carpule comprises a hollow body, a plug, and a sealing membrane;
the carpule being slidably engaged with the chamber;
the plug and the sealing membrane being positioned opposite each other along the hollow body;
the plug being positioned adjacent to the plunger;
the plug being slidably engaged with the hollow body, wherein movement of the plunger imparts corresponding movement to the plug;
the needle traversing into the hollow body through the sealing membrane;
the plunger comprises an elongated portion and a handle;
the handle being adjacently connected to the elongated portion;
the elongated portion traversing into the chamber; and
the elongated portion being slidably engaged with the chamber.

2. (canceled)

3. (canceled)

4. The improved anesthetic syringe as claimed in claim 1 comprises:
a conduit;
the conduit being positioned in the elongated body between the first opening and the chamber;
the first opening being in fluid communication with the chamber through the conduit; and
the needle traversing through the first opening, through the conduit, and into the chamber.

5. (canceled)

6. The improved anesthetic syringe as claimed in claim 1 comprises:
the enclosure further comprises a grip enhancement;
the grip enhancement being adjacently connected to the elongated body; and
the grip enhancement being positioned adjacent to the second opening.

7. The improved anesthetic syringe as claimed in claim 6 comprises:
the grip enhancement comprises a first brace and a second brace; and
the first brace and the second brace being positioned opposite each other around the elongated body.

8. The improved anesthetic syringe as claimed in claim 1 comprises:
the enclosure further comprises a window; and
the window being positioned on the elongated body, wherein the chamber is visible through the window.

9. (canceled)

10. An improved anesthetic syringe comprises:
an enclosure;
a plunger;
a carpule;
a needle;
a conduit;
the enclosure comprises an elongated body, a chamber, a first opening and a second opening;
the first opening and the second opening being positioned opposite each other along the elongated body;
the chamber being positioned within the elongated body between the first opening and second opening;
the carpule being housed within the chamber;
the plunger traversing into the chamber;
the plunger being positioned adjacent to the carpule;
the needle being in fluid communication with the carpule;
the needle being connected to a coupling body;
the needle being encircled by the coupling body;
the conduit being positioned in the elongated body between the first opening and the chamber;
the first opening being in fluid communication with the chamber through the conduit;
the needle traversing through the first opening, through the conduit, and into the chamber;
the carpule comprises a hollow body, a plug, and a sealing membrane;
the carpule being slidably engaged with the chamber;
the plug and the sealing membrane being positioned opposite each other along the hollow body;
the plug being positioned adjacent to the plunger;
the plug being slidably engaged with the hollow body, wherein movement of the plunger imparts corresponding movement to the plug; and
the needle traversing into the hollow body through the sealing membrane.

11. The improved anesthetic syringe as claimed in claim 10 comprises:
the coupling body being attached to the elongated body;
the needle traversing through the first opening into the chamber; and
the needle being positioned adjacent to the carpule.

12. The improved anesthetic syringe as claimed in claim 10 comprises:
the first opening and the coupling body each comprise a thread pattern; and
the thread pattern of the first opening being helically engaged with the thread patter of the coupling body.

13. (canceled)

14. The improved anesthetic syringe as claimed in claim 10 comprises:
the enclosure further comprises a grip enhancement;
the grip enhancement comprises a first brace and a second brace;
the grip enhancement being adjacently connected to the elongated body;
the grip enhancement being positioned adjacent to the second opening; and
the first brace and the second brace being positioned opposite each other around the elongated body.

15. The improved anesthetic syringe as claimed in claim 10 comprises:
the enclosure further comprises a window; and
the window being positioned on the elongated body, wherein the chamber is visible through the window.

16. The improved anesthetic syringe as claimed in claim 10 comprises:
the plunger comprises an elongated portion and a handle;
the handle being adjacently connected to the elongated portion;
the elongated portion traversing into the chamber; and
the elongated portion being slidably engaged with the chamber.

17. An improved anesthetic syringe comprises:
an enclosure;
a plunger;
a carpule;
a needle;
a conduit;
the enclosure comprises an elongated body, a chamber, a first opening and a second opening;
the plunger comprises an elongated portion and a handle;
the first opening and the second opening being positioned opposite each other along the elongated body;
the chamber being positioned within the elongated body between the first opening and second opening;
the carpule being housed within the chamber;
the plunger traversing into the chamber;
the plunger being positioned adjacent to the carpule;
the handle being adjacently connected to the elongated portion;
the elongated portion traversing into the chamber;
the elongated portion being slidably engaged with the chamber;
the needle being in fluid communication with the carpule;
the needle being connected to a coupling body;
the needle being encircled by the coupling body;
the conduit being positioned in the elongated body between the first opening and the chamber;
the first opening being in fluid communication with the chamber through the conduit;
the needle traversing through the first opening, through the conduit, and into the chamber;
the coupling body being attached to the elongated body;
the needle traversing through the first opening into the chamber;
the needle being positioned adjacent to the carpule;
the first opening and the coupling body each comprise a thread pattern; and
the thread pattern of the first opening being helically engaged with the thread patter of the coupling body.

18. (canceled)

19. The improved anesthetic syringe as claimed in claim 17 comprises:
the carpule comprises a hollow body, a plug, and a sealing membrane;
the carpule being slidably engaged with the chamber;
the plug and the sealing membrane being positioned opposite each other along the hollow body;
the plug being positioned adjacent to the plunger;
the plug being slidably engaged with the hollow body, wherein movement of the plunger imparts corresponding movement to the plug; and
the needle traversing into the hollow body through the sealing membrane.

20. The improved anesthetic syringe as claimed in claim 17 comprises:
the enclosure further comprises a grip enhancement and a window;
the grip enhancement comprises a first brace and a second brace;
the grip enhancement being adjacently connected to the elongated body;
the grip enhancement being positioned adjacent to the second opening;
the first brace and the second brace being positioned opposite each other around the elongated body; and
the window being positioned on the elongated body, wherein the chamber is visible through the window.
